(19) United States
(12) Patent Application Publication
(10) Pub. No.: US 2021/0379413 A1
(43) Pub. Date: Dec. 9, 2021

(54) DENTAL CONTAMINATE SHIELD

(71) Applicant: Dental PPE, LLC, Basking Ridge, NJ (US)

(72) Inventors: Preetmohinder S. Bagga, Basking Ridge, NJ (US); Charanpreet S. Bagga, Basking Ridge, NJ (US)

(21) Appl. No.: 17/341,717

(22) Filed: Jun. 8, 2021

Related U.S. Application Data
(60) Provisional application No. 63/035,966, filed on Jun. 8, 2020.

(51) Int. Cl. A62B 7/12; A61C 19/00; A62B 18/02; A62B 18/00; A62B 18/08

(52) U.S. Cl. CPC A62B 7/12; A61C 19/00; A62B 7/02; A62B 18/006; A62B 18/08; A62B 18/025

(57) ABSTRACT

A contaminate shield for protecting dentists and patients from harmful emissions during dental procedures is disclosed. The contaminate shield can include apertures allowing the dentist to access the patient's mouth through the contaminate shield. Further, the contaminate shield can include an aperture connectable to an air exchange system that provides clean air to the patient or removes contaminated air expelled by the patient. Additionally, the contaminate shield can be secured to the patient's face such that it covers the patient's nose and mouth to provide a physical barrier between the patient and a dentist.

### CROSS-REFERENCES TO RELATED APPLICATIONS

This application claims priority to U.S. Provisional Application No. 63/035,966, filed Jun. 8, 2020, the content of which is incorporated herein by reference in its entirety.

### BACKGROUND

During dental work, a dentist performs various kinetic activities in a patient's open mouth, such as drilling, cleaning, polishing, and rinsing. These activities can emit materials from the patient's mouth onto the dentist. The activities can also emit aerosolized particles into the air. If the patient currently has an infectious disease, the emissions can transmit the disease to the dentist. Moreover, because some emissions can linger in the air for a significant time and travel substantial distances, the disease may be transmitted to others in and around the examination room even after the dental procedure is completed.

### SUMMARY

The following presents a simplified summary of the claimed subject matter in order to provide a basic understanding of some aspects of the claimed subject matter. This summary is not an extensive overview of the claimed subject matter. It is not intended to identify key or critical elements of the claimed subject matter, or to delineate the scope of the claimed subject matter. Its sole purpose is to present some concepts of the claimed subject matter in a simplified form as a prelude to the more detailed description that is presented later.

The present disclosure generally relates to dental accessories, and more specifically to a contaminate shield for dental procedures. Implementations consistent with the present disclosure provide a contaminate shield that captures emissions during dental procedures. In some implementations, the contaminate shield includes an aperture connectable to an air exchange system that can provide fresh air to the patient or capture contaminated air emitted by the patient. Additionally, in some implementations, the contaminate shield can be secured to the patient's face such that it covers the patient's nose and mouth to provide a physical barrier between the patient and dentist. Further, in some implementations, the contaminate shield can include one or more apertures allowing the dentist to access the patient's mouth through the contaminate shield.

### BRIEF DESCRIPTION OF THE DRAWINGS

FIG. 1 shows a front-perspective view illustrating an example contaminate shield in accordance with aspects of the present disclosure.

FIG. 2 shows a side-perspective view illustrating an example contaminate shield in accordance with aspects of the present disclosure.

FIG. 3 shows an example environment in accordance with aspects of the present disclosure.

Particular implementations of the disclosed contaminate shield are illustrated in the drawings and described below. However, it is understood that the disclosed implementations are merely exemplary and may have various forms consistent with the present disclosure. Therefore, specific details disclosed herein are not to be interpreted as limiting, but merely as a basis for the claims and as a representative basis for teaching one skilled in the art to variously implement the concepts of the present disclosure.

### DETAILED DESCRIPTION

The present disclosure is directed to a system that protects patients and dentists from potentially harmful or contagious emissions during dental procedures. In some implementations the system comprises an air exchanger that captures air from the contaminate shield, which may be contaminated by the emissions from the patient and exhausts the potentially contaminated air to an external space. Additionally, in some implementations, an air exchanger can provide clean air from an external space to a patient via the contaminate shield to bypass potentially contaminated air within an examination room. Further, in some implementations, the system comprises a contaminate shield securable to a patient's face that protects dentists by blocking large particles and droplets emitted from the patient's mouth.

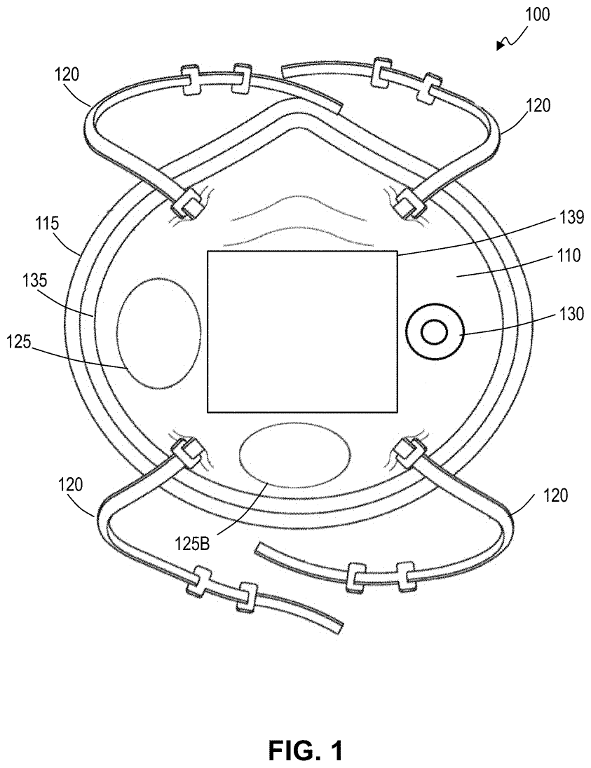

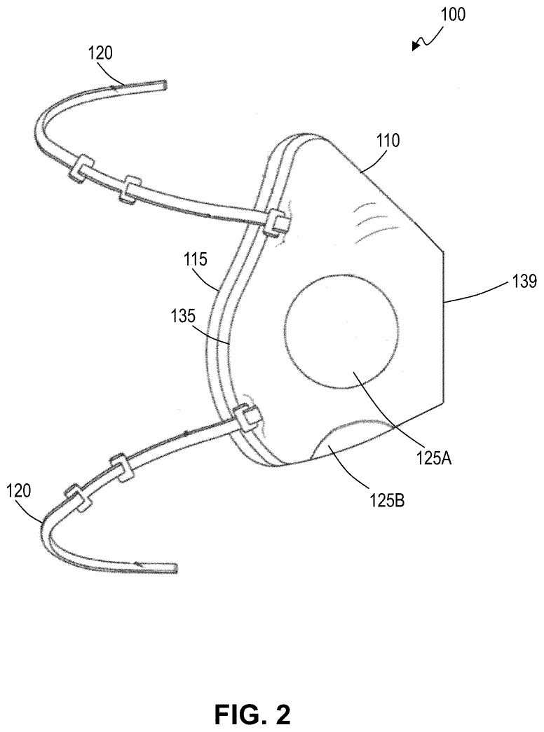

FIGS. 1 and 2 illustrate an example of a contaminate shield 100 in accordance with aspects of the present disclosure. FIG. 1 shows a front-perspective view of the ace shield 100 and FIG. 2 shows a side-perspective view of the contaminate shield 100. The contaminate shield 100 can include a mask body 110, a facial seal 115, and one or more straps 120. As illustrated in FIGS. 1 and 2, the mask body 110 can include one or more access apertures 125A and 125B, an air aperture 130, a peripheral edge 135, and a central area 139.

The mask body 110 can be generally cup-shaped. In some implementations, the mask body 110 can be contoured to receive oral and nasal features of a patient's face. In some implementations, the mask body 110 is constructed from a rigid, semi-rigid, or elastomeric material. For example, the material can be a plastic, such as polyvinylchloride, acrylic, polycarbonate, polyethylene, polystyrene, low density polyethylene (LDPE), isopyrene polymer, or the like. In some implementations, the material of the mask body 110 is transparent. Further, in some implementations, the material is a medical-grade plastic that is non-porous, anti-microbial, and sterilizable.

The apertures 125A, 125B, and 130 can be holes passing entirely through the mask body 110. In some implementations, the access apertures 125A, 125B and the air aperture 130 are positioned outside of the center area 139 of the contaminate shield 100. For example, the apertures 125A, 125B, and 130 are located adjacent to the peripheral edge 135 and do not intersect with the central area 139. Accordingly, the center area 139 can be a solid, uninterrupted barrier encompassing an area corresponding to a region directly in front of the patient's mouth and configured to shield the dentist from the emissions by blocking particles, droplets, aerosols, and the like ejected outward from the patient's mouth.

The access apertures 125A, 125B provide access to the patient's mouth for the dentist to perform procedures through the mask body 110. For example, the dentist may pass dental instruments through access aperture 125A and a suction instrument through access aperture 125B. In some implementations, the access aperture 125A can be positioned on the left or right side of the mask body 110. Additionally, in some implementations, the access aperture 125B can be located at a bottom of the mask body 110. Further, in some implementations, the access apertures 125A and 125B can have circular, oval, funnel, or other suitable shapes for passing dental instruments through the mask body 110. The diameter of the access apertures 125A and 125B can between about ½-inch to about ¾-inch.

In some implementations, the air aperture 130 has substantially the same size, shape, and position opposite the access apertures 125A or 125B on the mask body 110 such that the access aperture 125A or 125B and the air aperture 130 can be functionally interchangeable. In accordance with aspects of the present disclosure, the air aperture 130 is connectable to an air exchange system. In some implementations, the air exchange system can provide fresh or clean air via the contaminate shield 100 form an external space, which can be substantially isolated from the examination room. In some other implementations, the air exchange system can maintain negative pressure between the mask and the patient's mouth so as to capture emissions generated during a procedure. The negative pressure can provide sufficient draw to capture aerosolized particles. In some implementations, the air exchange system is a dental high volume evacuation (“HVE”) system having negative flow of about 100 cubic-feet per minute. By maintaining the negative pressure, the patient contaminate shield 100 protects the dentist during the procedure and protects others from emissions (e.g., aerosolized particles) that may linger after the procedure.

In some implementations, the air aperture 130 includes a coupler that mates with a corresponding coupler of an air conduit of the air exchanger. For example, the coupler can be a quick-release connector for pneumatic hoses. In some other implementations, the air aperture 130 can be sized and shaped to snugly fit a standard dental suction instrument. For example, the air aperture 130 can be a flexible gooseneck that receives a typical dental suction instrument. In some such implementations, the air aperture 130 can hold the suction instrument in a user-selectable position. For example, the air conduit can be a self-supporting, articulating arm or boom allowing multi-axial positioning of the suction instrument and contaminate shield 100 in a selected orientation.

In some implementations, the peripheral edge 135 of the mask body 110 is contoured to correspond to a shape of a person's face. For example, the contours of the peripheral edge 135 can correspond to the general shape of a patient's nose bridge, cheeks, mid to lower mandible, and chin. In some implementations, peripheral edge 135 can lay on the patient's face in contact with its contours, but without establishing an air-tight seal. In some implementations, the facial seal 115 provides a flexible layer between the peripheral edge 135 and the patient's face. The facial seal 115 can be a pliable gasket material, such as soft silicone or polymeric that lays in substantial contact with patient's skin around the perimeter of the peripheral edge. In some other implementations the facial seal 115 or the peripheral edge 135 can be covered with an adhesive that temporarily attaches the mask body 110 to the patient's face and is removable without harming the patient's skin. For example, the adhesive can be a skin-friendly adhesive, such as SOFTWEAR® provided by Adhesives Research of Glen Rock, Pa.

In some implementations, the central area 139 can be a substantially flat region of the mask body 110 located within the peripheral edge 135 and between the one or more access apertures 125A and 125B, and the air aperture 130. In some implementations, the central area 139 is substantially centered on the vertical axis, the horizontal axis, or both the horizontal and vertical axes of the contaminate shield 100. In some implementations, the central area 139 is a substantially flat, transparent, non-distorting plane of material providing a window through the contaminate shield 100 and allowing the dentist a substantially unobstructed and undistorted view of the patient's mouth during a procedure.

In some implementations, the mask body 110 includes one or more straps 120 that secure the mask body 110 to the patient's face. For example, the bands can be a pair of elastic loops that stretch behind the patient's ears. In other implementations the bands can be one or more elastic or length-adjustable bands that extend around the patient's head.

It should be understood that the contaminate shield 100 illustrated in FIGS. 1 and 2 is an example and that other arrangements, orientations, spacing, and sizing of the illustrated elements can be implemented in accordance with aspects of the present disclosure. For example, in some implementations, the contaminate shield 100 can be provided in left-handed or right-handed options for access to the left or right sides. Also, in some implementations, the shape of the patient contaminate shield 100 can be reversible and symmetrical, such that patient contaminate shield 100 can be rotated 180 degrees or flipped to allow access from left or right side. In some implementations, the contaminate shield 100 may come in several sizes and fit options depending upon the anatomic variations in patients, as well as preferences.

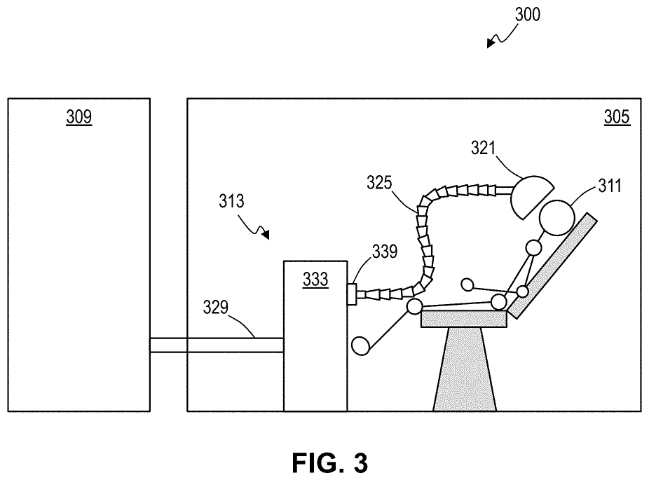

FIG. 3 shows an example environment 300 in accordance with aspects of the present disclosure. The environment 300 includes an interior space 305 and an exterior space 309 connected by an air exchange system 313. The interior space 305 can be an a substantially closed volume that may be occupied by one or more individuals, such as a room in a building. In accordance with some implementations, the interior space 305 can be an examination room for a dental patient 311.

The exterior space 309 can be a source of fresh or clean air that is substantially isolated from the interior space 305. In some implementations, the exterior space 309 is open, outdoor air. In some other implementations the exterior space 309 is a replaceable reservoir of clean or fresh air. For example, the source can be a tank of clean air or oxygen.

The air exchange system 313 can include a contaminate shield 321, a first air conduit 325, a second air conduit 329, and an air pump 333. The contaminate shield 321 can be a generally cup-shaped body. In some implementations, the contaminate shield 321 can be the same or similar to those previously described herein (e.g., contaminate shield 100).

In some implementations, the first air conduit 325 and the second air conduit 329 can be tubular ducts that convey gases, droplets, aerosols, floating particulates, and the like. The first air conduit 325 can connect the contaminate shield 321 to the air pump 333. For example, a first end of the first air conduit 325 can couple with an aperture of the contaminate shield 321 (e.g., air aperture 130) and a second end of the first air conduit 325 can be connected to the air pump 333.

In some implementations, a connector 339 between the second end of the first air conduit 325 and the air pump 333 supports the combined weight of the first air conduit 325 and the contaminate shield 321. Additionally, in some implementations, the connection 329 enables the first air conduit 325 to pivot in at least one-dimension, such as horizontally. Further, in some implementations, the connector 339 enables the first air conduit 325 to pivot and rotate in at least three-dimensions. Further, in some implementations, the connector 339 can maintain a selected position of the first conduit 325. For example, the connector 339 can include a friction bearing or a ratchet mechanism to retain a position on or adjacent to a face of the patient 311.

In some implementations, the first air conduit 325 can be a semi-rigid articulable hose bendable in two or more directions and, after positioning, maintain the position and orientation of the contaminate shield 321 in the interior space 305. For example, the first air conduit 325 can be comprised of a number of rigid tubular sections connected by friction washers or the like, all of which can be housed within a substantially air-tight skin.

The air pump 333 can be any type of device for generating negative or positive air pressure differential. In some implementations, the air pump 333 can be an air compressor or a vacuum pump. For example, the air pump 333 can include, for example, diaphragm compressor, an axial-flow fan, a propeller fan, a centrifugal fan, a mixed flow fan, a vane fan, or other suitable device. In some implementations, the air pump 333 can force exhaustion of air from the interior volume the contaminate shield 321 to the exterior space 309. In some implementations, the air pump 333 can be selectively switch between pushing air into the contaminate shield 321 and sucking air from the contaminate shield 321.

In accordance with aspects of the present disclosure, the air exchange system 313 can transfer fresh air between the contaminate shield 321 within the interior space 305 and the exterior space 309. In some implementations, the air exchange system 313 intakes (e.g., pulls or sucks) air from the interior space 305 via the contaminate shield 321 and exhausts the air to the exterior space 309. By doing so, the air exchange system 313 can minimize contamination of air within the interior space 305 when, for example, the patient 311 is infected a contagious respiratory disease. In some implementations, the air exchange system 313 can intake clean or fresh or clean air from the exterior space 309 and provides it the patient 311 within the interior space 305 via the contaminate shield 321. By doing so, the air exchange system 313 can bypass airflow to the patient 311 from the interior space 305 to prevent recirculating air within the interior space 305 to the patient 311. By doing so, the system avoids exposing the patient 311 to potentially contaminated air in the interior space 305.

It should be understood that the environment 300 illustrated in FIG. 3 is an example and that other arrangements, combinations, and orientations of elements can be implemented in accordance with aspects of the present disclosure. For example, the environment 300 is not limited to dental procedure and can be implemented in other interior spaces 305 where procedures generate potentially harmful gases and particles, such as medical offices or repair shops. Additionally, in some implementations, the environment 300 can include two or more contaminate shields 321, air conduits 325 and 329, or air pumps 333. For example, an interior space 305 can include multiple workstations having respective air exchange systems 313. Also, for example, a single workstation can include multiple contaminate shields 321 and air conduits 325 connected to a single air pump 333.

The present disclosure is not to be limited in terms of the particular implementations described in this application, which are intended as illustrations of various aspects. Moreover, the various disclosed implementations can be interchangeably used with each other, unless otherwise noted. Many modifications and variations can be made without departing from its spirit and scope, as will be apparent to those skilled in the art. Functionally equivalent methods and apparatuses within the scope of the disclosure, in addition to those enumerated herein will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular implementations only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

A number of implementations of the invention have been described. Various modifications may be made without departing from the spirit and scope of the invention. For example, various forms of the flows shown above may be used, with steps re-ordered, added, or removed. Accordingly, other implementations are within the scope of the following claims.

What is claimed is:

1. A contaminate shield comprising:
a body comprising a plurality of apertures,
wherein the plurality of apertures include:
one or more access apertures configured to receive one or more instruments, and
an air aperture configured to connect to an air exchange system.

2. The contaminate shield of claim 1, wherein the body comprises:
a mask contoured to receive oral and nasal features of a face; and
a peripheral edge having contours corresponding to a shape of the face.

3. The contaminate shield of claim 1, wherein the one or more access apertures comprise one or more thru-holes corresponding to a location of a mouth of the patient.

4. The contaminate shield of claim 3, wherein the one or more access apertures have diameters between about ½-inch and about ¾-inch.

5. The contaminate shield of claim 3, wherein the air aperture comprises a coupler configured to mate with a first air conduit.

6. The contaminate shield of claim 5, wherein the first air conduit comprises a semi-rigid articulable hose.

7. The contaminate shield of claim 6, wherein the mask body is configured maintain negative pressure between the mask and a mouth of the patient.

8. The contaminate shield of claim 1, wherein:
the body comprises a substantially flat region located within a peripheral edge of the mask body and between the one or more access apertures and the air aperture; and
the substantially flat region comprises a transparent, non-distorting plane of material.

9. A system comprising:
a contaminate shield;
an air exchanger;
a first air conduit connecting an interior space to the air exchanger via the contaminate shield; and
a second air conduit connecting the air exchanger to an exterior space,
wherein the exterior space is isolated from the interior space.

10. The system of claim 9, wherein the contaminate shield comprises:
one or more first apertures configured to receive one or more dental instruments, and
a second aperture configured to couple with the first air conduit.

11. The system of claim 9, wherein in the air exchanger is configured to:
intake clean air via the second air conduit; and
provide the clean air via the first air conduit and the contaminate shield.

12. The system of claim 11, wherein a direction of the air exchanger is reversible.

13. The system of claim 11, wherein the air exchanger is configured to:
maintain negative pressure between the contaminate shield and a mouth of the patient;
intake air from the interior space via the contaminate shield and the first air conduit; and
exhaust the air from the interior space into the exterior space.

14. The system of claim 11, wherein the first air conduit comprises a semi-rigid, articulable hose.

15. The system of claim 14, wherein the first air conduit is configured to maintain a selected position of the contaminate shield.

16. The system of claim 15, wherein the first air conduit is self-supporting.

17. The system of claim 9, wherein the volume of clean air is an outdoor air space.

18. The system of claim 9, wherein the volume of clean air is a replaceable reservoir.

19. The system of claim 9, wherein the first air conduit comprises:
a first end connected to a coupler of the contaminate shield; and
a second end connected to a connector of the air exchanger.

20. The system of claim 19, wherein the connector of the air exchanger is configured to pivot and rotate in three dimensions.
